(19) United States
(12) Patent Application Publication
(10) Pub. No.: US 2002/0106331 A1
(43) Pub. Date: Aug. 8, 2002

(54) Use of electrolytes (ions in solution) to suppress charging of inhalation aerosols

(76) Inventors: Joan Rosell, Castro Valley, CA (US); Igor Gonda, San Francisco, CA (US); Jeffrey Schuster, Berkeley, CA (US); Kui Liu, Redwood, CA (US)

Correspondence Address: Karl Bozicevic, BOZICEVIC, FIELD & FRANCIS LLP, Suite 200, 200 Middlefield Road, Menlo Park, CA, 94025, US

(21) Appl. No.: 09/733,610

(22) Filed: Dec. 8, 2000

(51) Int. Cl. A61L 9/04

(52) U.S. Cl. 424/45

(57) ABSTRACT

Formulations are disclosed as are aerosols created therefrom. The formulations are comprised of (a) a pharmaceutically active drug which does not ionize in solution; (b) an electrolyte; and (c) a solvent which is preferably water and/or ethanol. The electrolyte reduces electrostatic charging on particles of aerosol formed thereby enhancing characteristics of the aerosol particles which are important for efficient, repeatable intrapulmonary drug delivery.

### FIELD OF THE INVENTION

This invention relates generally to formulations and aerosols as well as dry powders created therefrom which are delivered to patients by inhalation.

### BACKGROUND OF THE INVENTION

Aerosol charging occurs in most aerosol generation methods, for example in spraying and in dry powder resuspension. (Hinds W C, Aerosol Technology, 2nd ed., Wiley-Interscience, 1999, section 15.4; John W, Particle Charge Effects, in Generation of Aerosols and Facilities for exposure experiments, K. Willeke ed., Ann Arbor Science, 1979) Often the charge is so high that experimental aerosols are neutralized by mixing them with gaseous ions. This requires equipment (radioactive sources, high voltage gas ionizers) that would be unsafe and impractical for portable therapeutic inhalers. In the development of portable inhalation systems, electrostatic charging is of concern because charging may cause: (a) aerosol deposition inside the device (resulting in decreased and more variable delivery efficiency), (b) aerosol deposition in the oropharynx, (c) electrical potential differences between user and device that could result in discomforting electric shocks to the user, (d) in applications targeting the deep lung, premature loss of particles in the upper and central airways.

The formulations and aerosols of the present invention endeavor to mediate these disadvantages.

### SUMMARY OF THE INVENTION

Formulations are disclosed which are comprised of (a) a pharmaceutically active drug which drug is not an electrolyte; (b) an electrolyte; and (c) a solvent. The invention further comprises aerosols of such formulations which have a particle size suitable for inhalation and to methods of treating patients by having them inhale such aerosols into their lungs.

An aspect of the invention is an aerosolized formulation of particles which generally have a particle size range suitable for inhalation (e.g. about 0.5 to about 10 microns) where the formulation is comprised of a solvent having an electrolyte and a drug dissolved and/or dispersed therein.

An aspect of the invention is a formulation which can be aerosolized to particles for inhalation without creating an excessive electrostatic charge which charge interferes with the delivery of the particles.

Another aspect of the invention is a formulation of water and/or ethanol having dissolved therein an electrolyte and a non-ionizable drug.

A feature of the invention is that a wide range of physiologically acceptable electrolytes can be used.

An advantage of the invention is that reduced electrostatic charge results in reduced attraction of the aerosolized particles to surfaces encountered prior to reaching the user's lungs.

Another aspect of the invention is that aerosolized particles of formulation can create dry powders by evaporating away the solvent, and such dry powders will not have the excessive electrostatic charge that would cause a range of problems including their deposition in the manufacturing equipment and hence cause manufacturing losses.

Yet another aspect of the invention is specific formulations of electrolytes in solutions of ethanol and drugs which are substantially insoluble in water.

Another feature of the invention is that water and various combinations of water and ethanol can be used as the solvent.

Another feature of the invention is propellants (such as low boiling point propellants) that contain nonionizable drugs dissolved in them with or without the use of co-solvents, the co-solvents being, for example, ethanol, water or mixtures of ethanol plus water.

In another aspect of the invention the drug is suspended in such propellants in which case the drug could be an electrolyte but because of the low concentration of the ionized drug dissolved in the propellant, the ionized drug by itself would not effectively prevent charging of the aerosol droplets during the aerosolization process, thus requiring the addition of an electrolyte.

Still another aspect of the invention is a method of reducing the electrostatic charge on particles of aerosol created for inhalation.

These and other objects, advantages, and features of the invention will become apparent to those persons skilled in the art upon reading the details of the invention as more fully described below.

### BRIEF DESCRIPTIONS OF THE DRAWINGS

FIG. 1 is a graph of data obtained from Example 1 of net aerosol charge versus run number.

FIG. 2 is a graph of data obtained from Example 2 of net aerosol charge versus run number.

FIG. 3 is a graph of data obtained from Example 3 of net aerosol charge versus run number.

FIG. 4 is a graph showing the effect of emitted dose from adding different concentrations of sodium chloride to the formulation.

### DETAILED DESCRIPTION OF PREFERRED EMBODIMENTS

Before the present formulations, aerosols and methods are described, it is to be understood that this invention is not limited to particular formulations, aerosols or methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a non-electrolytic drug” includes a plurality of such drug and reference to “an electrolyte” includes reference to one or more electrolytes and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The term “electrolyte” means any substance which, if dissolved in water or another solvent, will provide ionic conductivity to the resulting solution. Preferred electrolytes of the invention are non-toxic to humans and are present in a amount sufficient to reduce and more preferably eliminate electrostatic charge on particles of aerosol formed from the formulation. Preferred electrolytes are readily soluble in water and/or ethanol and include salts generally found in humans such as sodium and potassium chloride.

The term “non-electrolytic drug,” “non-ionizable drug,” and the like are used interchangeably here to mean any drug which when dissolved in water and/or ethanol or in a formulation containing another suspension medium or solvent does not readily form positive and negative ions. Such a drug may be solid or liquid at room temperature (e.g. 18°-25° C.) and may have any degree of solubility in a suitable solvent.

The terms “particle diameter” and “diameter” are used when referring to the diameter of an aerosol particle and are defined as the “aerodynamic diameter”. The “aerodynamic diameter” is the physical diameter of a sphere of unit density (1 gm/cm³) that has the same terminal sedimentation velocity in air under normal atmospheric conditions as the particle in question. This is pointed out in that it is difficult to accurately measure the physical diameter and density of small particles using current technology and because the shape and density may be continually changing as may its size due to factors such as evaporation and surrounding humidity. In addition, the deposition of aerosol particles in the bronchial airways of a human subject is described by a Stokes impaction mechanism which is characterized by a particle's aerodynamic diameter. Thus, the diameter of one particle of material of a given density will be said to have the same diameter as another particle of the same material if the two particles have the same terminal sedimentation velocity in air under the same conditions.

The term “liquid” is used here to describe any composition which would generally be described as a liquid under the temperature and pressure conditions it is used. Thus, “liquid” includes water, ethanol and mixtures thereof which are liquid at STP, but also includes low boiling point propellants such as hydrocarbons, halocarbons for example chlorofluorocarbons (CFCs) and hydrofluoroalkanes (HFAs) which are gaseous at STP but are liquid when held in a canister at high pressure (see U.S. Pat. No. 5,910,301). The liquid may be any solvent or may be a carrier liquid for a dispersion of small particles which are substantially insoluble in the liquid.

Abbreviations: LC=Label claim amount; mM=millimolar, NaCl=Sodium Chloride.

### Invention in General

The invention includes various aspects such as formulations, aerosols created from formulations, as well as methods of creating aerosols and dry powders for inhalation. The different aspects of the invention have in common the use of an electrolyte in order to reduce or eliminate detectable electrostatic charge on particles of the aerosol or dry powder.

Aerosolized formulations of the present invention can have particles of any diameter. However, the particles preferably have a diameter which is suitable for inhalation by a patient and such diameter is generally in the range of about 0.5 micron to about 10 microns, more preferably 1 micron to about 5 microns and still more preferably about 2 microns to about 4 microns. The formulation is comprised of an electrolyte, a non-ionizable drug and a solvent. The electrolyte and the drug may both be dissolved completely within the solvent. Alternatively, the drug may be dispersed in the solvent in the form of fine dispersion which dispersion has particle sizes which are the same as or less than the particle size of the particles or aerosol created for inhalation.

The electrolyte may be an alkali halide of any type such as sodium chloride or potassium chloride and is preferably a material which is non-toxic and physiologically compatible with the internal surfaces of a patient's lungs. Electrolytes can be a halide of an alkali earth metal such as calcium chloride or may be an inorganic salt or acid thereof such as hydrochloric acid, sulfuric acid, phosphoric acid or any of the pharmaceutically acceptable salts thereof provided the acid or salt thereof is present in the formulation in a sufficiently dilute concentration so as to not cause harm to the internal linings of the patient's respiratory tract. Other electrolytes include compounds such as ammonium hydroxide, acetic acid, sodium acetate, ascorbic acid, as well as salts of ascorbic acids such as sodium salts. Further, the electrolyte may be an organic acid, organic base or pharmaceutically acceptable salts of such acids or bases. Those skilled in the art will understand from this disclosure that a wide range of different compounds may be used as the electrolyte and that mixtures of electrolytes can be used. Preferred electrolytes vary somewhat with the liquid used in the formulation.

The drug may be any drug. However, the essence of the invention is emphasized by formulations which consist only of non-ionizable drugs in the medium in which they are dissolved or suspended, i.e. do not include substantial amounts of drugs which form ions when dissolved in water or other solvents. Suitable non-ionizable drugs can be any drug currently known or later developed which is not ionizable when dissolved in water or other solvents. Useful examples of such drugs include the following: Amphotericin; Estrone; Antiviral drugs, e.g. Ribavirin; Fluticasone propionate; Beclomethasone dipropionate; Hexamethyl melamine; Benzodiazepines; Lorazepam; Budenoside; Phentanyl base; Cyclosporin; Retinoids; Diazepam; Surfactant protein; Droperidol; Testosterone; Ergotamine; THC and its derivatives; Estradiol; Triamcinolone acetonide. The examples also include proteins, peptides and gene vectors, such as inhalable particles containing them dispersed in a propellant.

The electrolyte may be present in any concentration which is sufficient to decrease and more preferably substantially eliminate electrostatic charge on particles of aerosol created using the formulation. It is believed that the electrolyte should be present in the formulation in a concentration of about 10¹⁹ ions per liter or more or more preferably 5×10²⁰ ions per liter or more.

The solvent may be any solvent. However, water and ethanol are preferred solvents. With some drugs which are not ionizable it is difficult to dissolve the drug in water. Accordingly, ethanol or mixtures of ethanol and water are suitable for such drugs. A range of different compounds including alcohols such as isopropyl alcohol, glycerol, propylene glycol, polyethylene glycols which are generally known as solvents can be used as solvents in connection with the present invention. Even solvents that may not be preferred to due to potentially adverse physiological effects such as methanol, ketones such as acetone, esters, dimethylsulfoxide can be used when the particles are formed to allow complete or near complete evaporation of the solvent before being taken up by the patient. What is meant by non-ionizable is that when the drug is present in the formulation under the conditions it will be used at, it is not ionized or not ionized in an amount such that it has a substantial effect on the electrostatic charge of particles from the formulation as compared to the effect on electrostatic charges obtained by the electrolyte e.g. less than 1/100 the effect caused by the electrolyte. Alternatively, the drug may be ground into a fine powder and dispersed in the solvent or carrier liquid thereby creating a suspension.

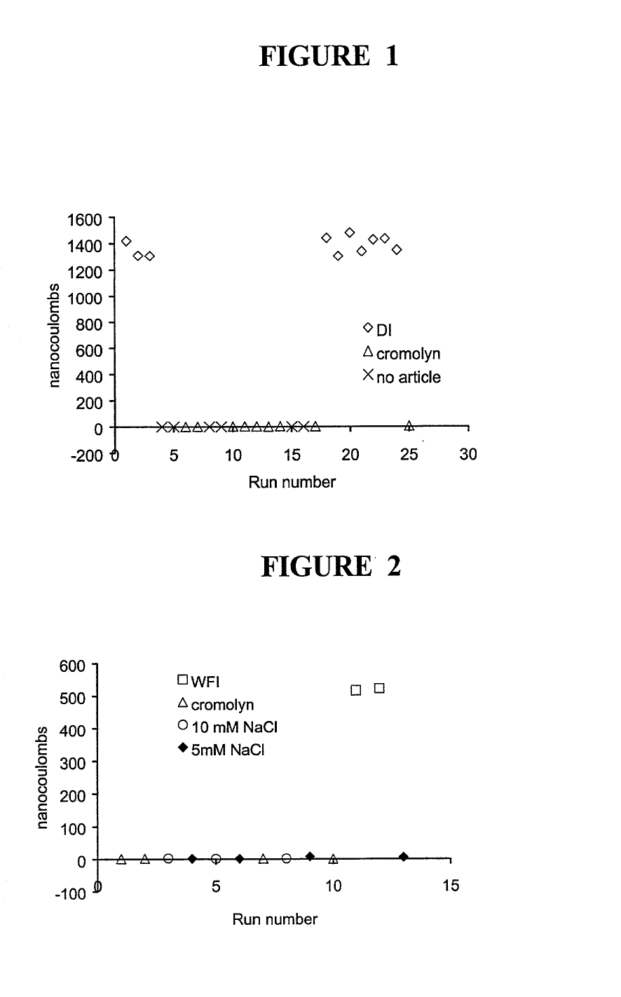

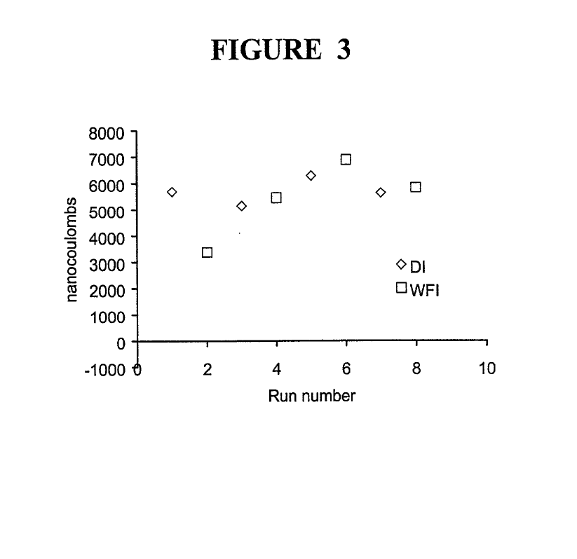

Formulations of the invention can be aerosolized into fine particles in a manner which allows the solvent or carrier liquid to evaporate quickly leaving substantially dry particles. The dry particles can then be accumulated and then used in a dry powder inhaler (DPI) device and delivered to patients by inhalation. However, the invention is preferably used in connection with devices where the liquid formulation is moved through small pores in a flexible porous membrane of the type disclosed in U.S. Pat. Nos. 5,544,646 and 6,123,068 which are incorporated herein by reference. By moving the formulation through the small pores of the porous membrane streams will exit the pores and the streams will disassociate into particles which are substantially uniform in size. In the absence of an electrolyte the particles formed will have a significant electrostatic charge as shown in FIGS. 1, 2 and 3. However, by the inclusion of an electrolyte in the formulation the charge on the particles is decreased or, as shown in FIGS. 1, 2 and 3, reduced to very low levels.

A basic formulation of the invention is comprised of an electrolyte, a non-ionized drug and a liquid. The liquid is preferably a solvent which has both the electrolyte and the drug dissolved therein. However, the liquid may be a carrier liquid which has the drug dispersed therein. A small amount of solvent may be added to the carrier liquid in order to allow the electrolyte to form ions thereby making it possible to decrease or substantially eliminate electrostatic charge.

Examples of non-ionizable drugs which drugs are non-ionized within a formulation of the invention include the following: Amphotericin; Estrone; Ribavirin; Fluticasone propionate; Beclomethasone dipropionate; Hexamethyl melamine; Benzodiazepines; Lorazepam; Budenoside; Albuterol; Salmeterol; Fentanul; Phentanyl base; Cyclosporin; Retinoids; Diazepam; Surfactant protein; Droperidol; Testosterone; Ergotamine; THC and its derivatives; Estradiol; Triamcinolone acetonide.

There are commercially available drugs and drugs which have yet to be developed which do not form ions in a non-aqueous medium. In accordance with the present invention these drugs can be ground into a fine powder or produced in a fine powder form by technology known to those skilled in the art and thereafter dispersed in a non-aqueous liquid. Drugs which fall into this category include peptides such as insulin, insulin analogs, momomeric insulin, lispro insulin, and a wide range of proteins which have either local or systemic effects. Useful proteins include human growth hormone, various growth factor proteins, erythropoeitin, alpha-, beta-, and gamma-inteferons, antibodies used therapeutically or diagnostically could be formulated in such a non-aqueous medium as can soluble receptors, cytokines, amylin, various synthetic proteins or chemically modified proteins such as pegylated proteins including pegylated alpha inteferon, parathyroid hormone, and calcitonin.

In addition to the basic components of the liquid, drug and electrolyte the formulation of the invention may include a range of additional components which are used to provide some additional characteristics to the formulation. These additional components may be present in any desired amount and should be present in an amount sufficient to enhance a characteristic of a formulation. Often, such components are present in very small amounts such as less than 10% by weight, more preferably less than 5% and still more preferably less than 1%. Examples of such additional components include components such as a solubilizer, a stabilizer, a pH adjuster, a buffer and an osmolarity adjuster. When the liquid is a dispersing medium for a drug suspension it is preferable to include a surfactant. The formulation may also include small amounts of materials such as antimicrobial compounds which are not intended as drugs to have an antimicrobial effect on the patient but rather to prevent the growth of microorganisms within the formulation.

A formulation of the present invention may be comprised of an electrolyte, a propellant and a pharmaceutically active drug which does not provide a substantial effect on the electrostatic charge of the formulation. The liquid is generally a compound selected from the group consisting of hydrocarbon, a halocarbon, a chlorocarbon, a fluorocarbon, a chlorofluorocarbon, a chlorofluorohydrocarbon, a perfluorocarbon, a hydrofluoroalkane, an ether, a ketone, a dimethylsulfoxide and mixtures thereof.

When the formulation is comprised of a propellant as the liquid the formulation preferably includes a small amount of solvent which dissolves the electrolyte thereby enabling the electrolyte to form ions within the formulation.

The formulations of the invention are preferably designed for intrapulmonary drug delivery. Thus the formulations are designed so that they can form aerosols wherein the aerosols have a particle size in the range of about 0.5 to 10 microns and more preferably 1 to 5 microns and still more preferably about 2 microns to about 4 microns. Although the invention is not limited to such the following provides some specific examples of formulations and tests on the those formulations demonstrating how the formulations of the invention make it possible to decrease or substantially eliminate detectable levels of electrostatic charge.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

The AERx™ system (as described and disclosed in U.S. Pat. Nos. 5,544,646 and 6,123,068) produces aerosols by extruding the liquid contents of a single dose blister packet (here 45 uL) through an array of micro-drilled holes. The jets formed during the extrusion process are entrained by the patient inhalation air flow. (Schuster J et al., In Drug Delivery to the Lungs VIII, pp14-17, 1997; Schuster J et al., In Respiratory Drug Delivery VI, pp. 83-90, 1998).

For the measurements of electric charge, the air flow was simulated with pressurized house air. A Faraday cup was inserted in the flow path of the aerosol a few centimeters downstream from the point of generation. It comprised a perforated aluminum cartridge filled with a paper filter, which was inserted into and insulated from a grounded metal enclosure, used as a shield from external electromagnetic noise. A coaxial wire connected to the aluminum cartridge was passed through the metal enclosure and connected to a current-voltage converter of the op-amp design (ammeter). The voltage output from the ammeter was acquired as a function of time by a computer together with other information about the extrusion process, such as the position of the piston that pressurizes the blister pack during an extrusion.

The formulations tested in EXAMPLES 1, 2 and 3 were de-ionized water (DI), sterile water for injection USP (WFI), 5 mM and 10 mM sodium chloride in water, and 30 mg/ml sodium cromoglycate (cromolyn) in water.

The region between aerosol generation and the Faraday cup was inspected after each extrusion (“run”), and any residue found was wiped clean. This region was all metallic in the system used here and comprised two separable parts, the so-called “clamp”, in which the packet is held and the aerosol is generated, and the so-called “diffuser”, which is positioned immediately downstream from the clamp. This diffuser was removed in some of the Examples herein discussed. The conditions for Examples 1, 2 and 3 are summarized in table 1. Several tests were preformed in order to validate our technique, namely to show that the measured currents were indeed associated with the aerosol (see summary in table 2).

TABLE 1
Experimental conditions

| EXAMPLE | FORMULATIONS | DIFFUSER |
|---|---|---|
| #1 | DI, cromolyn | On |
| #2 | WFI, cromolyn, 5 & 10 mM NaCl | Not present |
| #3 | WFI, DI | Not present |

TABLE 2
Summary of technique validation tests

| TEST | RESULT |
|---|---|
| Empty packets | Current at noise level |
| Air flow interrupted mid extrusion | Current dropped to noise level |
| Add mouthpiece and glass throat | Signal shifted in time by 0.1-0.2 |
| between generation and detection | seconds, as expected |
| Cromolyn aerosol collection in a | Cromolyn assay results were |
| filter downstream of Faraday cage | below quantitation limit |

### Results

A measure of the net electric charge carried by the bolus was obtained from the numerical integral over time of the electric current signals. In FIGS. 1, 2 and 3, corresponding to EXAMPLES 1, 2 and 3 respectively, the net charge thus obtained is shown as a function of run number for various formulations.

In EXAMPLE 1 (FIG. 1), aerosols from DI water carried a large positive charge, while those filled with cromolyn produced a significantly reduced charge level. Several runs carried out with a “dummy” packet that contained no liquid (labeled “no article” by the “X” in FIG. 1) gave rise to no measurable levels of charge. DI water consistently gave rise to a much greater residue in the diffuser than cromolyn, for which the residue was a small fraction of the initial packet contents. The difference in residue is another indication of high charging. It should be noted that only the aerosol that did not contribute to the residue could contribute to the charge measured, and therefore, that the charging at generation in the case of DI water is likely to have been much more than measured.

In EXAMPLE 2, both NaCl and cromolyn solutions produced much lower charge than water for injection. Because no diffuser was used in these tests, the residues found after each run were small, including the case of WFI. In summary, the results from EXAMPLES 1 and 2 suggest that small amounts of electrolyte can produce a dramatic reduction in aerosol electrostatic charge.

In EXAMPLE 3, DI and sterile water led to similar charging. Interestingly, the charge was several times higher in EXAMPLE 3 than in EXAMPLES 2 and 3. In the case of DI water, this was in part because the diffuser was removed in EXAMPLE 3 as compared to EXAMPLE 1 (and less residue was found this time as a result). However, the disagreement from WFI cannot be explained in this way. If, as found in EXAMPLES 1 and 2, low amounts of electrolytes can have a dramatic effect on the resulting charge, it is conceivable that some variability in the levels of impurities in the DI water and WFI may have contributed to the variability in charge seen across the different experiments.

### Conclusions

AERx™ aerosols of pure water have a high charge. These are associated with deposition of the aerosol near the point of aerosol generation, presumably due to the strong self-repulsion of the aerosol cloud. Small amounts of electrolyte suppressed both such effects. The high efficiency of delivery from the AERx™ system deep into the lung (53-80% of loaded dose,) (Farr S et al., Int. J. Pharm. 198, 63-70, 2000; Smaldone G C et al., J. Aer. Med. 12(2), 98, 1999) provides clear evidence that when using formulations which contain an electrolyte, electrostatic charge effects do not play a significant role in the system performance.

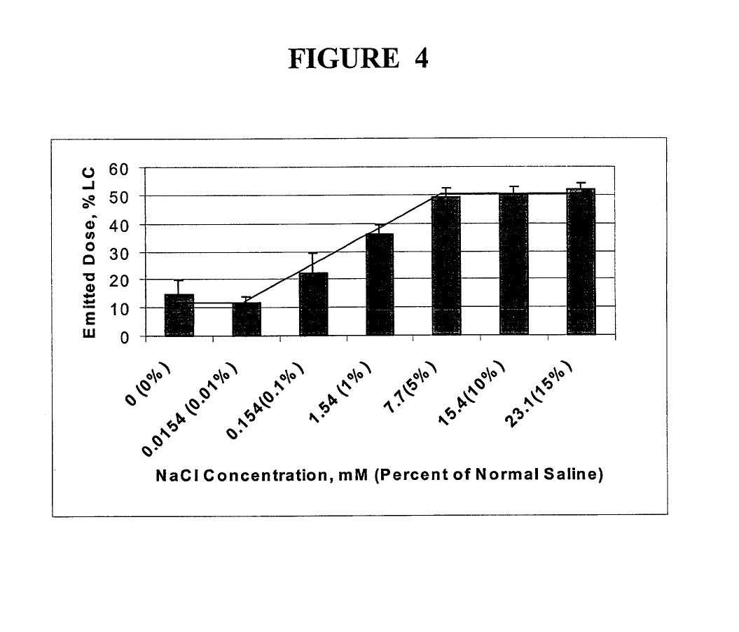

Effect on emitted dose of adding sodium chloride to an ethanol-water mixture containing a non-ionizable drug are shown in FIG. 4. The aerosol is produced using an AERx™ device loaded with single dose dosage forms containing 50 microliters of this formulation. The solvent is an 80% by volume ethanol-water mixture. At each concentration, the aerosol from each of a number of dosage forms was collected and chemically assayed. Results shown in the graph of FIG. 4. The emitted dose is low and variable at zero and low concentrations of electrolyte, due to precipitation of the aerosol inside the device. As more sodium chloride is added, the emitted dose increases until at sufficiently high concentrations of this electrolyte, the emitted dose reaches a plateau value in which electrostatic effects disappear.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. An aerosolized formulation of particles having an aerodynamic diameter in a range of about 0.5 micron to about 10 microns, comprising:
an electrolyte;
a non-ionized drug; and
a liquid.

2. The aerosolized formulation of claim 1, wherein the liquid is a solvent and the electrolyte and drug are dissolved in the solvent.

3. The aerosolized formulation of claim 1, wherein the particles have an aerodynamic diameter in a range of about 1 micron to about 5 microns.

4. The aerosolized formulation of claim 1, wherein the liquid is a carrier liquid and the drug is dispersed in the carrier liquid.

5. The aerosolized formulation of claim 1, wherein the particles have an aerodynamic diameter in a range of about 2 microns to about 4 microns.

6. The aerosolized formulation of claim 1, wherein the electrolyte is an alkali halide.

7. The aerosolized formulation of claim 6, wherein the alkali halide is selected from the group consisting of sodium chloride and potassium chloride.

8. The aerosolized formulation of claim 1, wherein the electrolyte is a halide of an alkali earth metal.

9. The aerosolized formulation of claim 8, wherein the electrolyte is calcium chloride.

10. The aerosolized formulation of claim 1, wherein the electrolyte is selected from the group consisting of inorganic acids and salts thereof.

11. The aerosolized formulation of claim 10 wherein the electrolyte is selected from the group consisting of hydrochloric acid, sulfuric acid, phosphoric acids and salts of any of the acids.

12. The aerosolized formulation of claim 1, wherein the electrolyte is selected from the group consisting of ammonium hydroxide, acetic acid, sodium acetate, ascorbic acid and a sodium salt of ascorbic acid.

13. The aerosolized formulation of claim 1, wherein the electrolyte is selected from the group consisting of an organic acid, an organic base, a salt of the organic acid, and a salt of the organic base.

14. The aerosolized formulation of claim 1, wherein the non-ionizable drug is selected from the group consisting of Amphotericin; Estrone; Ribavirin; Fluticasone propionate; Beclomethasone dipropionate; Hexamethyl melamine; Benzodiazepines; Lorazepam; Budenoside; Albuterol; Salmeterol; Fentanyl; Phentanyl base; Cyclosporin; Retinoids; Diazepam; Surfactant protein; Droperidol; Testosterone; Ergotamine; THC and its derivatives; Estradiol; Triamcinolone acetonide.

15. The aerosolized formulation of claim 1, wherein the electrolyte is present in a concentration of about 10¹⁹ ions per liter or more.

16. The aerosolized formulation of claim 1, wherein the electrolyte is present in a concentration of about 5×10²⁰ ions per liter or more.

17. The aerosolized formulation of claim 1, wherein the liquid is comprised of water.

18. The aerosolized formulation of claim 1, wherein the liquid is comprised of ethanol.

19. The aerosolized formulation of claim 1 wherein the liquid is selected from the group consisting of water, an alcohol, an alkane, a glycol, a glycerol and mixtures thereof.

20. The aerosolized formulation of claim 1, wherein the liquid is a solvent selected from the group consisting of water, ethanol and a mixture of water and ethanol and wherein the electrolyte is dissolved in the solvent.

21. The aerosolized formulation of claim 1, wherein the formulation further comprises an additional component selected from the group consisting of a solubilizer, a stabilizer, a pH adjuster, a buffer, and an osmolarity adjuster.

22. The aerosolized formulation as claimed in claim 1, wherein the liquid is a solvent and the formulation further comprises a surfactant.

23. The aerosolized formulation of claim 1, wherein the liquid is a carrier liquid and the non-ionized drug is dispersed therein and the formulation further comprises a stabilizing agent.

24. The aerosolized formulation as claimed in claim 1, wherein the formulation is further comprised of an osmolarity adjuster, a pH stabilizing agent, and a fluid density adjuster.

25. An aerosolized formulation of particles having an aerodynamic diameter in a range of from about 0.5 micron to about 10 micron, comprising:
an electrolyte;
a non-ionized drug; and
a solvent having the drug and electrolyte dissolved therein, wherein the electrolyte is present in a concentration in a range of about 10¹⁹ ions per liter or more.

26. An aerosolized formulation of particles having an aerodynamic diameter in a range of about 0.5 to 10 microns, comprising:
an electrolyte;
a propellant; and
a pharmaceutically active drug characterized by not providing a substantial effect on electrostatic charge of the particle as compared to an effect on electrostatic charge obtained from the electrolyte.

27. A method producing dry powder particles, comprising:
aerosolizing a formulation comprising an electrolyte, a non-ionized drug and a liquid to form an aerosol of particles;
allowing the liquid to evaporate from the particles and form dry particles; and
collecting the dry particles.

28. The method of claim 27, wherein the liquid is a solvent selected from the group of water and ethanol and the dry particles have a diameter of about 0.5 to about 10 microns.

29. The method of claim 27, wherein the liquid is a compound selected from the group consisting of a hydrocarbon, a halocarbon, a chlorocarbon, a fluorocarbon, a chlorofluorocarbon, a chlorofluorohydrocarbon, a perfluorocarbon, a hydrofluoroalkane, an ether, a ketone, a dimethylsulfoxide and mixtures thereof.

30. The method of claim 27, wherein the formulation further comprises a solvent which dissolves the electrolyte thereby enabling the electrolyte to form ions within the formulation.

31. The aerosolized formulation of claim 4, wherein the non-ionizable drug is selected from the group consisting of Amphotericin; Estrone; Ribavirin; Fluticasone propionate; Beclomethasone dipropionate; Hexamethyl melamine; Benzodiazepines; Lorazepam; Budenoside; Albuterol; Salmeterol; Fentanyl; Phentanyl base; Cyclosporin; Retinoids; Diazepam; Surfactant protein; Droperidol; Testosterone; Ergotamine; THC and its derivatives; Estradiol; Triamcinolone acetonide.

32. The aerosolized formulation of claim 1, wherein the liquid is a non-aqueous solvent and the drug is selected from the group consisting of insulin, an insulin analog, monomeric insulin, and insulin lispro.

33. The aerosolized formulation of claim 1, wherein the liquid is a non-aqueous solvent and the drug is a protein.

34. The aerosolized formulation of claim 33, wherein the protein is selected from the group consisting of human growth hormone, human growth factor, erythropoeitin, alpha-, beta-, and gamma-inteferon, an antibody, a soluble receptor, a cytokine, amylin, pegylated protein, pegylated alpha inteferon, parathyroid hormone, calcitonin, folcile stimulating hormone, and alpha-1 antitrypsin.

35. The aerosolized formulation as claimed in claim 1, wherein the liquid is a non-aqueous solvent and the drug is a nucleotide sequence.
